(19) United States
(12) Patent Application Publication
(10) Pub. No.: US 2012/0189679 A1
(43) Pub. Date: Jul. 26, 2012

(54) METHOD FOR COATING POWDERS

(76) Inventors: Marilyn Calderone, Albi (FR); Elisabeth Rodier, Marsal (FR)

(21) Appl. No.: 13/404,935

(22) Filed: Feb. 24, 2012

Related U.S. Application Data
(62) Division of application No. 11/662,365, filed on Apr. 20, 2007.

(30) Foreign Application Priority Data
Sep. 9, 2004 (FR) 0409563

(51) Int. Cl. A61K 9/50; A61P 29/00; A61P 17/04; A61P 3/02; A61P 1/04; A61P 9/12; A61P 25/24; A61Q 17/04; A61K 8/11; A61P 31/00

(52) U.S. Cl. 424/401; 424/490

(57) ABSTRACT

The invention concerns a method for coating powdery solid active substances which includes the following steps: a) providing (2; 3; 4) a mixture comprising at least one coating agent wherein is dissolved a supercritical or subcritical fluid in conditions of temperature and pressure enabling said fluid to be maintained in supercritical or subcritical conditions; b) providing (7) individualized moving particles of at least one powdery solid active substance; c) contacting (8) the mixture and the moving individualized particles of active substance in conditions of temperature and pressure ensuring simultaneously expansion of the supercritical or subcritical fluid and its restoration to gaseous state, the atomization of the coating agent and its solidification around the moving individualized particles of active substance; d) separating and recuperating the coated particles (9). The invention also concerns coated microparticles, wherein the average size of the active substance core is less than 50 μm.

This is a divisional of, and claims priority to, Ser. No. 11/662,365, filed Mar. 8, 2007, which is the U.S. national phase of PCT/FR2005/002236, filed Sep. 8, 2005. Priority is also claimed to French Application No. 0409563, filed Sep. 9, 2004. The foregoing applications are hereby incorporated by reference.

The field of the invention is the coating of particles of solid active substances, in particular with sizes less than 100 μm and advantageously of less than 50 μm, by a process using a supercritical or subcritical fluid, and the manufacture of microcapsules.

The state of the art with regard to the technique for coating industrially comprises chemical processes, mechanical processes, that is to say, including only physical phenomena (J. Richard et al., Techniques de l'ingénieur [Techniques for the Engineer], Microencapsulation, J 2 210, 1-20, 2002), and physicochemical processes.

The chemical processes are based on the in situ formation of the coating material by polycondensation, polymerization, and the like. The mechanical processes employ spraying techniques (spray drying, spray coating), drop- or droplet-forming techniques (prilling) and extrusion techniques (extrusion, spheronization). Finally, the physicochemical processes are based on controlling the variation in the solubility and the conditions for the precipitation of the coating agents: pH, temperature, and the like.

Coacervation processes, which are limited to lipophilic active principles alone, are known among physicochemical processes. Solvent evaporation or extraction processes, which are easy to carry out, are also known. The main limits to these processes are that they apply only to the encapsulation of a lipophilic active material and in particular that they employ organic solvents. First, the products obtained have a not insignificant residual solvent content and, secondly, a drying stage is necessary to complete the coating of the particles.

Finally, the physicochemical processes also include microencapsulation by simple coacervation and by thermal gelling. The first process also employs organic solvents and the second, for its part, has the main disadvantage of being limited to active principles which are not heat-sensitive.

Chemical processes, such as interfacial polycondensation, emulsion polymerization, suspension polymerization, dispersion polymerization, and the like, which make it possible to manufacture in situ a polymer membrane at the surface of emulsion droplets by virtue of a chemical reaction between two monomers, are also known for coating. The chemical reaction takes place at the interface between the dispersed phase and the dispersing phase. This coating method applies only to solutions of active materials. It is therefore not applicable to the coating of solid forms.

The coating of solid forms is carried out according to processes which can be classified into two subgroups, mechanical processes and processes using the supercritical fluids technique.

The mechanical processes are based on spraying (spray drying or spray coating, fluidization), on formation of drops (prilling) or on an extrusion principle. The processes using spraying or the formation of drops involve the use of a liquid formulation composed of a coating agent dissolved in a solvent. The material to be coated can be in the liquid or solid form. The extrusion principle involves a molten medium composed of the molten coating agent mixed with a solid or molten material to be coated which has to be thermally stable at the extrusion temperature, generally between 70° C. and 150° C. The size of the microcapsules obtained by these processes is generally greater than or equal to 100 μm. These processes thus do not prove to be suitable for the coating of materials to be coated in solid forms having a size of less than 100 microns and in particular for heat-sensitive materials to be coated.

The processes using a compressed gas are generally based on the use of supercritical CO₂. This is because the latter possesses numerous advantages: relatively low critical coordinates (31° C., 7.47 MPa), great variation in its solvating power for low variations in pressures, its nontoxicity and its relatively low cost. The three main implementations with a view to coating powders are the RESS (Rapid Expansion of Supercritical Solution) technique, the PGSS (Particles from Gas Saturated Solution) technique and the SAS (Supercritical Anti Solvent) technique.

The RESS technique (Türk et al., Journal of Supercritical Fluids, 15, 1999, 79-89) makes it possible to coat particles, the particle size of which can currently fall down to 50 microns, using the technology of beds fluidized by a supercritical fluid. This process, controlled by the German team of Brunner (Journal of Supercritical Fluids, 24, 2002, 137-151), consists in fluidizing the particles which it is desired to coat with a fluid under supercritical conditions and in then, in this bed, using a spray nozzle, reducing in pressure a saturated supercritical phase over the fluidized particles, coating them. The extraction of the coating agent by the supercritical fluid takes place in a chamber separate from the fluidized bed. The difference in pressure between the extraction chamber and the fluidized bed brings about a sudden fall in the supersaturation and, thereby, the crystallization of the coating agent. However, this promising technique remains limited only to coating agents which are soluble in supercritical CO₂. The range of coating agents which can be envisaged thus remains highly restricted.

On the other hand, the solubility of compressed CO₂ in liquids and solids is generally very high. This principle has given rise to the PGSS process. The supercritical or subcritical fluid is here not therefore the solvent, as for the RESS process, but the solute. The main advantage of the PGSS process is thus that of greatly widening the panel of coating agents which can be processed with a compressed gas. The PGSS technique employed in WO 02/05944 and U.S. Pat. No. 6,056,791 is based on the dissolution of compressed CO₂ in the coating agent, the melting point or the glass transition temperature of which is thus lowered. The dissolution of the compressed gas in the coating agent brings about the softening thereof and then the reduction in pressure of this softened phase brings about the solidification of this material in the form of a pulverulent solid. For the coating of particles, the material to be coated is mixed with this molten medium. The material to be coated/coating agent combination is subsequently reduced in pressure, which brings about the solidification of the coating agent on the material to be coated. A major shortcoming of this coating process is that it is not suitable for heat-sensitive active principles since the coating agent and the material to be coated are placed under similar pressure and temperature conditions throughout the duration, generally lengthy, of the dissolution of the compressed gas in the coating agent. Moreover, the precise control of the material to be coated/coating agent ratio very often proves to be difficult.

Moreover, the CPF Technology (Concentrated Powder Form) process, based on the PGSS principle (WO 99/17868), is also known. It consists in cospraying a liquid active principle, pressurized beforehand, with a pulverulent auxiliary in order to obtain solid/liquid formulations. A gas under pressure is dissolved in a solution of active principle until saturation is achieved. This saturated solution is rapidly reduced in pressure through a nozzle, thus forming very fine droplets. During the expansion, a stabilizing powder is added cocurrentwise with the liquid spray. The expansion of the gas at the nozzle outlet brings about intensive mixing between the liquid drops resulting from the aerosol and the pulverulent solid added. The liquid of the spray is then adsorbed at the surface of the solid and solid/liquid particles are formed. The CPF process thus makes it possible to convert a liquid form to the solid form by adsorption on a solid support. One of the main applications relates to the improvement in the transportation and storage of the solid/liquid composite particles with respect to the starting liquid form. This technology also makes it possible to obtain novel solid/liquid composite particles according to the adsorption properties of the support solid. The aim desired here is to stabilize an active principle in the liquid form via a pulverulent product.

The SAS technique, applied to the coating of active principles (Patents U.S. Pat. No. 5,043,280 and FR 2 753 639), requires the dissolution beforehand of the coating agent in an organic solvent. The material to be coated is dispersed in this solution. This dispersion is subsequently coinjected with supercritical CO₂. The latter acts as antisolvent and thus makes it possible to solidify the coating agent on the material to be coated. One of the major constraints on the process is the use of an organic solvent, which subsequently has to be separated from the antisolvent, but also in particular the fact that the active principle remains, throughout the duration of the process, under pressure and temperature conditions which are very often high (and at least above 31° C. if CO₂ is used as supercritical fluid).

Foster et al. (Powder Technology, 126, 2002, 134-149) present a specific implementation of the antisolvent process by the ASES (Aerosol Solvent Extraction System) process. A spray nozzle for the coating of active principles makes it possible to deposit coating agents on the particles formed simultaneously by an antisolvent effect. This is because the nozzle developed comprises three concentric annular sections, making it possible to introduce the various fluids into a chamber under supercritical conditions. The solution of active principle dissolved in an organic solvent is conveyed at the centre. The central part of the nozzle makes it possible to convey the organic solution with the dissolved polymer and the external part of the nozzle makes it possible to convey the supercritical fluid which produces the antisolvent effect. Thus, the coating agent is deposited preferentially on the active principle in order to coat it. The coating agent and the material to be coated crystallize simultaneously. It should be noted that this type of material to be coated/coating agent contacting operation is carried out on streams of fluids and the nozzle does not at any point convey the active principle in the solid form.

Finally, the inventory of patents for coating by the supercritical route would not be complete if mention were omitted of Patent Application FR 2 809 309. It relates to the formation of microspheres of matrix type intended to be injected, comprising a protein active principle and an agent which coats this active principle. The coating is targeted at prolonging the release. The said coating agent has to be soluble in the supercritical fluid. The coated particles obtained are characterized in that they are devoid of organic solvent. The principle of this process consists in placing the active principle and the coating agent in an autoclave equipped with a stirrer and in then introducing, with stirring, a fluid under supercritical conditions, so that the latter dissolves the coating agent. The active principle remains solid throughout the process. Once the coating agent has dissolved, the temperature and the pressure of the suspension are slowly reduced and are controlled in order to bring about a sudden fall in the supersaturation of the coating agent in the supercritical phase. The coating agent is then deposited preferentially on the active principle and forms a protective coating layer. However, this process remains restricted to coating agents exhibiting sufficient solubility in the presence of a supercritical fluid.

In the light of the various techniques mentioned, it appears necessary to have available a process for coating smaller solid particles of heat-sensitive active substances with a broader range of coating agents and in particular with coating agents which are insoluble in supercritical or subcritical fluids.

To this end, the present invention relates to a process for coating pulverulent solid active substances, characterized in that it comprises the following stages:
a) providing a mixture comprising at least one coating agent in which is dissolved, up to saturation, a supercritical or subcritical fluid under temperature and pressure conditions which make it possible to maintain the said fluid under supercritical or subcritical conditions;
b) providing separate moving particles of at least one pulverulent solid active substance;
c) bringing the mixture and the separate moving particles of active substance into contact under temperature and pressure conditions which simultaneously ensure:
   the reduction in pressure of the supercritical or subcritical fluid and its return to the gaseous state,
   the spraying of the coating agent and
   its solidification around the separate moving particles of active substance;
d) separating and recovering the coated particles.

One of the advantages of the process according to the present invention is that, by virtue of the use of temperatures of approximately 25° C. and thus close to ambient temperature, the process according to the present invention is particularly suitable for the coating of heat-sensitive active substances: this is because, on the one hand, the active substance and the coating agent are treated separately and, on the other hand, the time for bringing the active substance and the coating agent into contact is very short, it additionally being possible for the characteristics of the coating agent to be modified during stage (a) (lowering of the melting point, for example).

The process as described by the present invention also makes possible good control of the active substance/coating agent ratio by weight within the range which can be between 10/1 and 1/10, advantageously between 5/1 and 1/5, and thus of the amounts of active substances and of coating agent involved.

Finally, since the principle of the process according to the present invention is based on the dissolution of a supercritical or subcritical fluid in the coating agent and not on the dissolution of the coating agent by the said fluid, it can be applied to a wide panel of coating agents and in particular to coating agents which are insoluble in the said fluid.

The term “coating” is understood, within the meaning of the present invention, as the deposition of a continuous layer of a coating agent on a particle of solid active substance. A structure of solid core/continuous solid shell type is thus obtained. The shell can be porous or nonporous. The particles obtained are therefore microcapsules.

The term “active substance” is understood, within the meaning of the present invention, as any pharmaceutical active principle (analgesics, antipyretics, aspirin and derivatives, antibiotics, antiinflammatories, antiulceratives, antihypertensives, neuroleptics, antidepressants, oligonucleotides, peptides or proteins, for example), cosmetic active principle (UV inhibitor or self-tanning agent, for example), nutraceutic active principle (vitamins, for example), food active principle, agrochemical active principle or veterinary active principle. Advantageously, it is pseudoephedrine. Advantageously, the active substance is heat-sensitive. The term “heat-sensitive active substance” is understood, within the meaning of the present invention, as any active substance which, under the action of temperature, experiences modification to its physicochemical structure. Advantageously, these substances are sensitive to temperatures of greater than approximately 28° C., advantageously of greater than approximately 25° C. Advantageously, they are proteins or peptides, advantageously BSA (Bovine Serum Albumin).

The term “coating agent” is understood, within the meaning of the present invention, as one or more coating material(s) deposited at the surface of the active substance and capable of forming a continuous solid layer around the solid particle of active substance. Advantageously, the coating agent is insoluble in the supercritical or subcritical fluid. Use will advantageously be made of fatty substances, such as phospholipids, in particular phosphatidylcholine, phosphatidylglycerol, diphosphatidylglycerol, dipalmitoylphosphatidylcholine or dioleoylphosphatidylethanolamine, triglycerides of capric and caprylic acids, esters of fatty acids which are solid, in particular their esters of C₈ to C₁₈ fatty acids, such as the ethyl palmitate, ethyl myristate or octyldodecyl myristate, advantageously esters of C₈ to C₁₈ fatty acids and their mixtures. The coating agents can also be polysaccharides and their derivatives, such as starch or modified starch, for example carboxymethylstarches; cellulose or modified ethylcellulose, for example carboxymethylcelluloses, ethylcellulose, hydroxyethylcellulose, hydroxypropylmethylcellulose, methylhydroxyethylcellulose or methylhydroxypropylcellulose; or polysaccharides resulting from alginate, from carrageenans, from pectin, from pectates, from guars, from xanthans and from chitosan; synthetic polymers of acrylic or methacrylic type, such as homopolymers or copolymers of acrylic or methacrylic acid, polyacrylamide, polycyanoacrylates and generally any synthetic polymer well known to a person skilled in the art derived from acrylic or methacrylic acid; vinyl polymers and copolymers derived from vinyl esters (poly(vinyl acetate)), copolymers of ethylene and of vinyl acetate; biodegradable polymers and copolymers of α-hydroxycarboxylic acids, in particular the homopolymers and copolymers of lactic and glycolic acids, more particularly PLA and PLGA; poly(ε-caprolactone) and its derivatives, poly(β-hydroxybutyrate), poly(hydroxyvalerate) and β-hydroxybutyrate-hydroxyvalerate copolymers, poly(malic acid); amphiphilic block polymers of poly(lactic acid), poly(ethylene oxide) type, biocompatible polymers of poly(ethylene glycol), poly(ethylene oxide)s type or block copolymers of poly(ethylene oxide), polypropylene oxide) type; polyanhydrides, polyorthoesters, polyphosphazenes and their mixtures.

The coating agent is advantageously chosen from the group consisting of polysaccharides and their derivatives, synthetic polymers of acrylic or methacrylic type, lipids, phospholipids or a mixture of these.

The coating agent is advantageously chosen from a mixture of mono-, di- and triglycerides, advantageously a glyceryl dipalmitate/stearate, such as Precirol® sold by Gattefossé, paraffin wax and glyceryl monostearate.

The coating agent advantageously makes it possible to mask the taste and/or the color of the active substance.

The coating agent advantageously makes possible controlled release of the active substance and an increase in its biodegradability.

More advantageously still, the coating agent protects the active substance with regard to external agents which cause damage.

In this case, the active substance is advantageously sensitive to heat and/or to chemicals and/or to light.

The term “supercritical or subcritical fluid” is understood, within the meaning of the present invention, as any fluid used at a temperature or a pressure greater than their critical value or at a temperature or a pressure lower than but close to their critical value. It is advantageously CO₂ but it can also be any gas capable of saturating the coating agent, such as, for example, butane, nitrogen or nitrous oxide.

The coating process of the present invention is a “mechanical” process since it employs a technique of spraying the coating agent over the separate moving particles of active substance. The said process relates to the coating of an active substance in the pulverulent solid form by a coating agent liquefied by a supercritical or subcritical fluid under temperature and pressure conditions which make it possible to maintain the said fluid under supercritical or subcritical conditions and then solidified by the reduction in pressure. The active substance and the coating agent are therefore both in the solid form in the final particles obtained by the process according to the present invention.

The process of the present invention thus involves two main stages: the dissolution of a supercritical or subcritical fluid in a coating agent and then the reduction in pressure of the coating agent/supercritical or subcritical fluid mixture when simultaneously brought into contact with the separate moving particles of active substance. It is also possible to envisage placing the fluid under supercritical or subcritical conditions after the said fluid has been mixed with the coating agent.

Furthermore, an organic solvent chosen from the group consisting of ketones, alcohols, esters, alkanes, alkenes and a mixture of these can be added to the separate moving particles of active substance and/or to the coating agent/supercritical or subcritical fluid mixture.

This is because it is possible to envisage coating nanopowders manufactured in situ with supercritical CO₂. In this case, the process requires the addition of a cosolvent. The particles to be coated are then dissolved in the said cosolvent and the solution is brought into contact with the coating agent and the supercritical or subcritical fluid.

Furthermore, the addition of a small amount of a solvent makes it possible to modify the solubility of the CO₂ in the coating agent and it is therefore possible to modify the throughputs of the mixture obtained while retaining the same coating characteristics (active substance/coating agent ratio).

Likewise, a plasticizing agent can be added to the coating agent/supercritical or subcritical fluid mixture. The plasticizer makes it possible to improve the surface qualities of the coating agent according to the application desired.

The first stage makes it possible to modify the physicochemical characteristics of the coating agent, for example by lowering the melting point, the glass transition temperature and the viscosity. The second stage involves the reduction in pressure of the mixture comprising the molten or softened coating agent and the fluid in order to form an aerosol: product in the micronized form. The lowering in temperature and in pressure thus generated makes it possible to solidify the coating agent around the separate moving particles of solid active substance, it being possible for these moving particles of active substance to be optionally already present in the chamber where the coating agent/fluid mixture is reduced in pressure or transported to and injected into the same chamber, where the contacting operation takes place, simultaneously with the reduction in pressure.

At the end of the process, the coated particles can be recovered in a gas/solid separation filter.

A fundamental characteristic of the process of the present invention is the fluidization of the particles of active substance to be coated, which makes it possible to present the said substance in the form of fine moving separate particles which are not agglomerated, and to thus coat the said separate moving particles of active substance with a size of less than 100 μm and advantageously of less than 50 μm, more advantageously still of less than 20 μm, and not aggregates of active material. The particles of active substance can be given motion in various ways known to a person skilled in the art: pneumatic, mechanical or by fluidized bed, for example.

In a specific embodiment of the invention, the separate particles of active substance are conveyed by pneumatic or mechanical transportation (with CO₂ or compressed air, for example). This is because this type of conveying makes it possible to fluidize the particles which will be coated and to prevent the aggregation thereof. In the case of pneumatic transportation, CO₂ will preferably be used as gas. Advantageously, this CO₂ can be recycled to make possible continuous reuse of the gases, reuse possible in the case where the supercritical or subcritical fluid dissolved in the coating agent is also CO₂.

In a specific embodiment of the invention, stage (c) of bringing the mixture into contact with the separate particles of active substance is carried out in a feed stream obtained by coinjection. Advantageously, this coinjection consists of the simultaneous injection through the coaxial pipe of the separate particles of active substance to be coated and of the coating agent/fluid mixture.

Advantageously, the feed stream is linear or helical.

The process according to the present invention can also be used in the case of coating several different active substances intended for the same final formulation.

The process according to the present invention applies to particles of active substance having a variable geometrical shape (regular shape: spherical or certain crystalline forms, for example; or any other irregular shape).

The present invention additionally relates to coated microparticles of active substance, each microparticle successively comprising a core comprising the active substance and a shell comprising the coating agent, characterized
in that they are capable of being obtained by the process according to the present invention,
in that the active substance is solid at ambient temperature and is sensitive to heat,
in that the mean size of the core of active substance is less than 50 μm, advantageously less than 20 μm.

The coating agent is advantageously solid at ambient temperature and is insoluble in the supercritical or subcritical fluid. The particles according to the present invention are advantageously devoid of organic solvent.

The present invention additionally relates to coated particles of active substance, each particle successively comprising a core comprising the active substance and a shell comprising the coating agent, characterized
in that they are capable of being obtained by the process according to the present invention,
in that the active substance is solid at ambient temperature and is sensitive to heat,
in that the mean size of the core of active substance is less than 100 μm,
in that the coating agent is solid at ambient temperature and is insoluble in the supercritical or subcritical fluid, and
in that the coated particles are devoid of organic solvent.

The coating agent advantageously makes it possible to mask the taste and/or the color of the active substance and/or makes possible controlled release of the active substance and an increase in its bioavailability and/or makes it possible to protect the active substance with regard to external agents which cause damage. The active substance is advantageously sensitive to chemicals and/or to light. The coating agent advantageously makes it possible to formulate two or more active substances which are incompatible in nature with one another and thus to protect them with respect to one another.

The present invention additionally relates to the use of the particles according to the present invention in an oral, topical, injectable or rectal formulation and/or in pharmaceutical, cosmetic, nutraceutic, food, agrochemical or veterinary formulations.

The invention will be better understood and the aims, advantages and characteristics of the invention will become more clearly apparent from the description which follows and which is made with reference to the appended drawings, representing nonlimiting implementational examples of the invention, in which:

FIG. 1 represents a first embodiment of the process according to the present invention with recirculation of the uncoated separate particles of active substance.

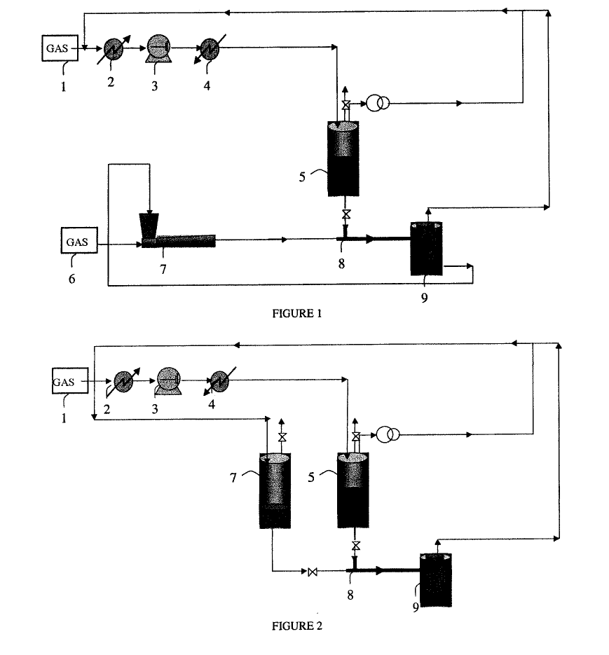

FIG. 2 represents a second embodiment of the process according to the present invention with recirculation of the CO₂, providing the two functions of supercritical or subcritical fluid dissolved in the coating agent in the autoclave and of carrier gas in the presence of separate particles of active substance to be coated.

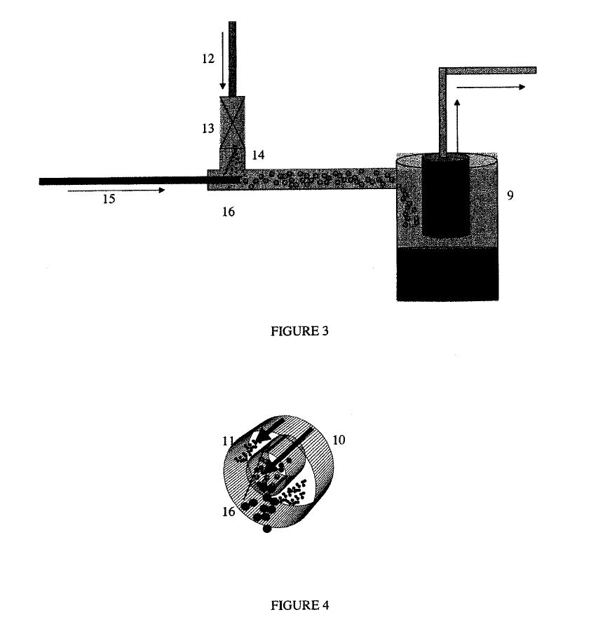

FIGS. 3 and 4 represent a method of coinjection of the separate moving particles of active substance and of the coating agent/supercritical or subcritical fluid mixture in concentric pipes.

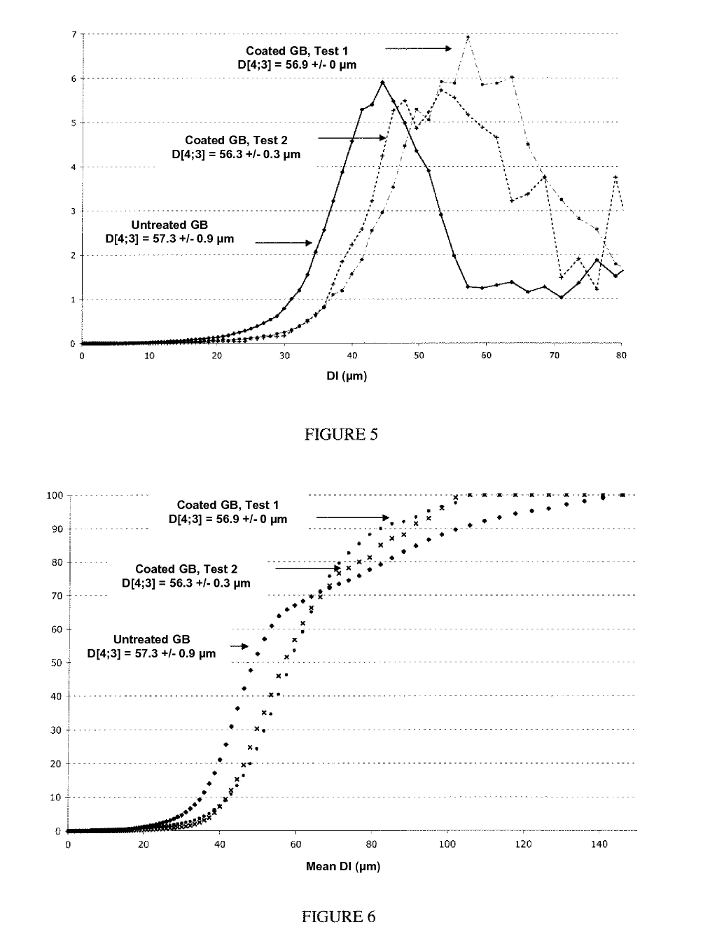

FIG. 5 represents the differential volume percentage obtained for uncoated glass particles or glass particles coated by the process according to the present invention.

FIGS. 6, 11 and 15 represent the cumulative distribution by volume for glass particles of different sizes which are coated with different coating agents according to the present invention or which are uncoated.

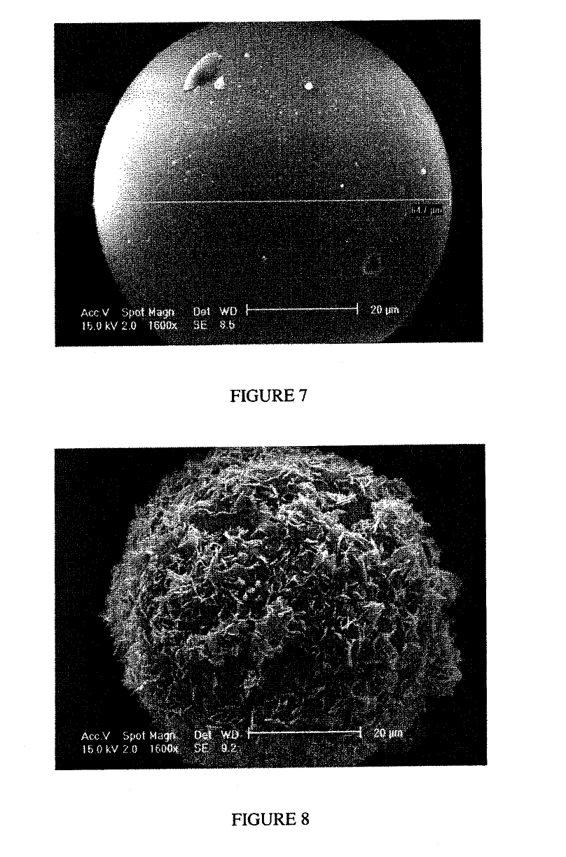

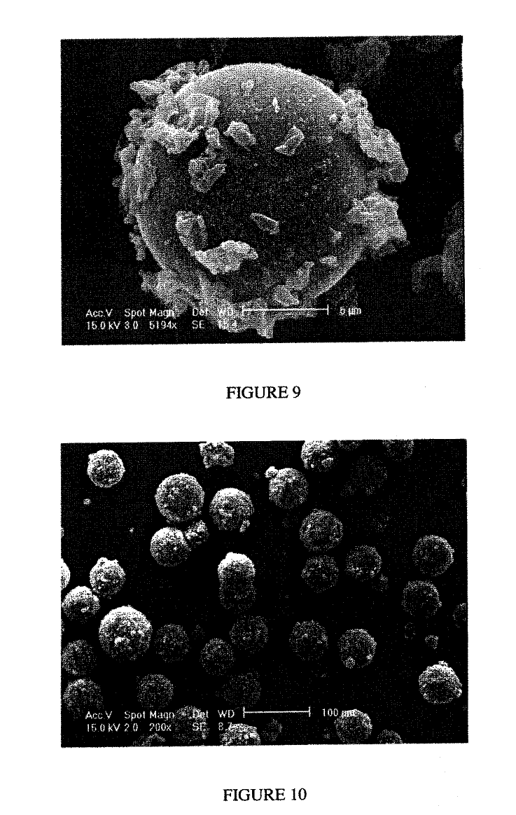

FIGS. 7 to 10 represent the images obtained with an ESEM (Environmental Scanning Electron Microscope) of uncoated micrometric glass beads (50-63 μm) (FIG. 7), micrometric glass beads coated with Precirol® ATO5 (FIGS. 8 and 10) and a physical mixture of these micrometric glass beads (50-63 μm) with Precirol® ATO5 (FIG. 9).

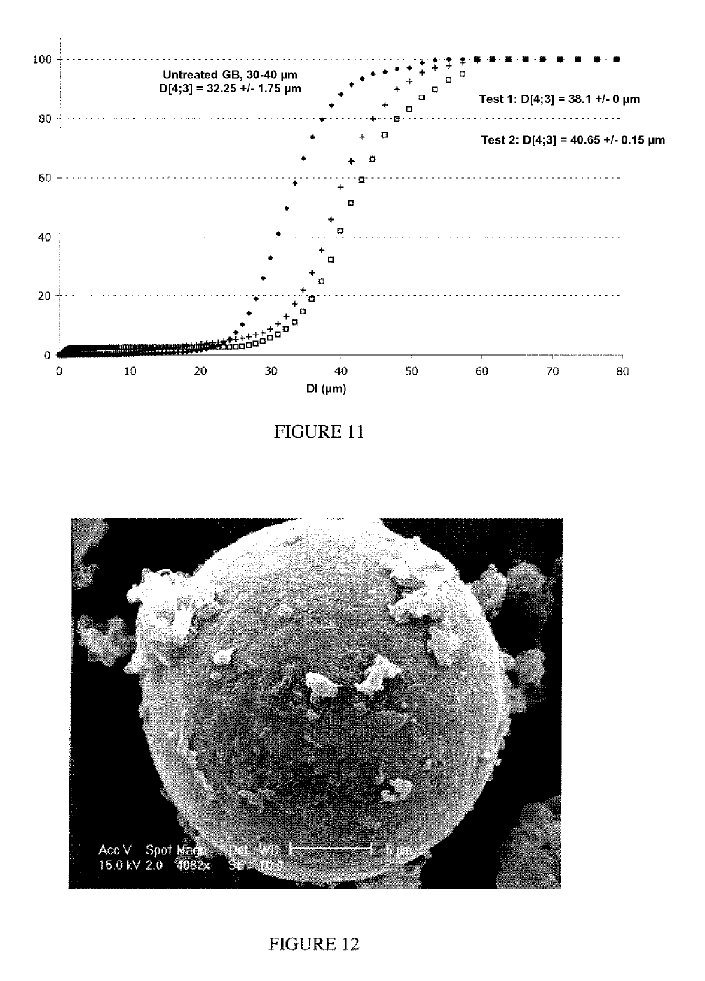

FIG. 12 represents the images obtained with an ESEM of micrometric glass beads (30-40 μm) coated with Precirol® ATO5.

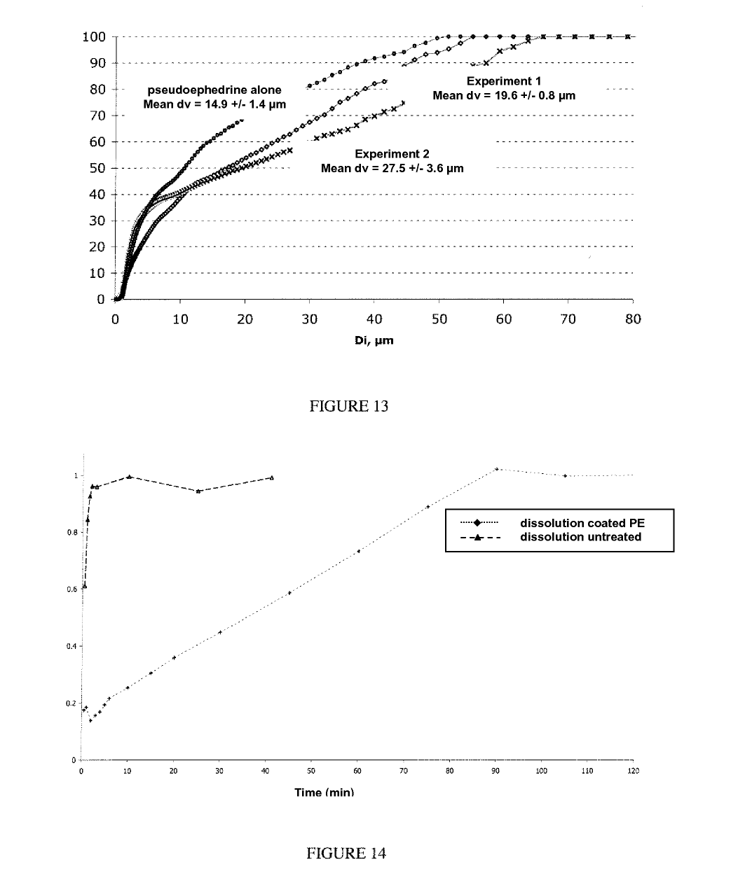

FIG. 13 represents the cumulative distribution by volume for coated particles of pseudoephedrine according to the present invention or uncoated particles of pseudoephedrine.

FIG. 14 represents a test of release in distilled water of uncoated particles of pseudoephedrine or coated particles of pseudoephedrine according to the present invention.

FIG. 16 represents the images obtained with an ESEM of glass beads with a particle size of less than 20 μm coated with paraffin wax.

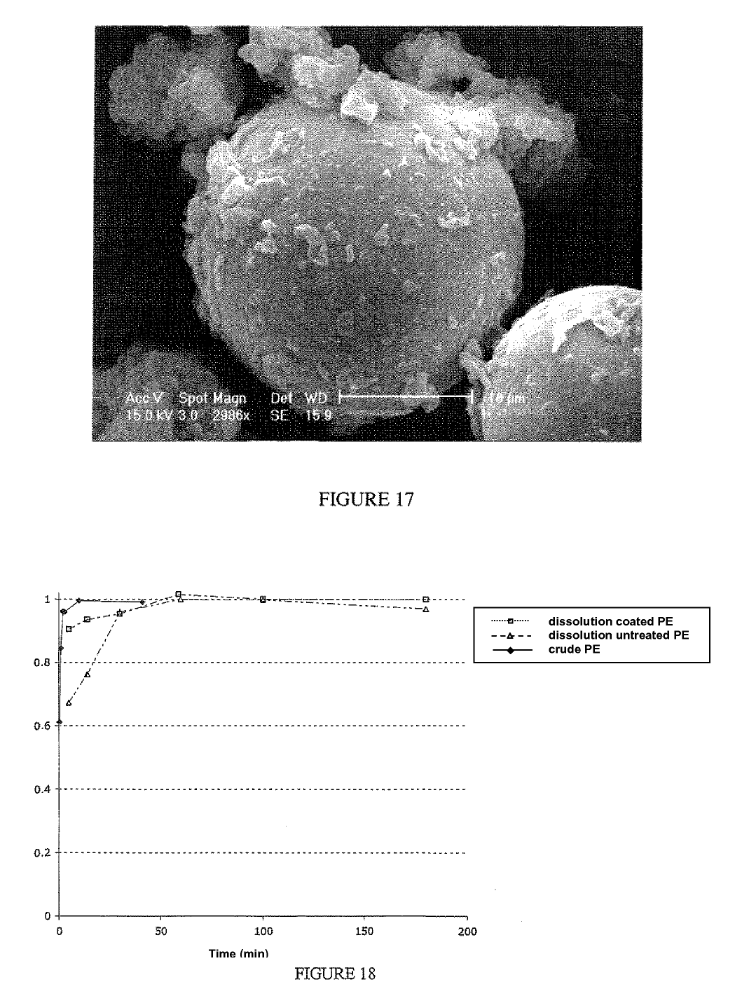

FIG. 17 represents the images obtained with an ESEM of glass beads with a particle size of less than 20 μm coated with glyceryl monostearate.

FIG. 18 represents a test of release into solution of uncoated particles of pseudoephedrine or coated particles of pseudoephedrine according to the present invention.

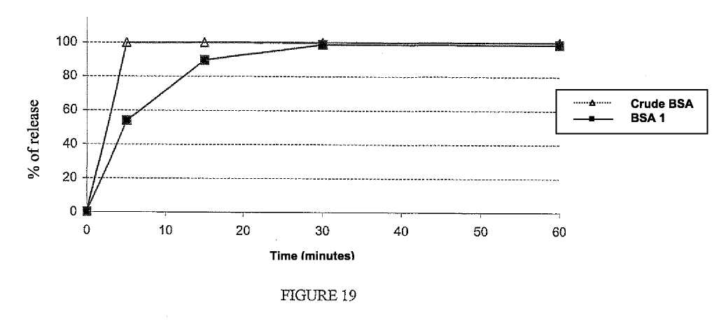

FIG. 19 represents a test of release into solution of uncoated particles of BSA proteins or coated particles of BSA proteins according to the present invention.

The process represented in FIG. 1 is carried out in the following way:

The fluid (CO₂) originating from a liquid stock (1) is cooled (2), then pumped (3) and heated via a heat exchanger (4). It then becomes supercritical or subcritical. The fluid is introduced under supercritical or subcritical conditions into the autoclave (5) comprising the coating agent. The temperature of the autoclave is adjusted according to the melting point of the coating agent at atmospheric pressure. Generally, the saturation of the coating agent by a compressed gas brings about a fall in the melting point which can range up to 40° C. below the melting point under standard conditions. The said fluid is dissolved in the coating agent until saturation is reached. During this period, the pressure is kept constant. On reaching equilibrium, the softened mixture of coating agent saturated with supercritical or subcritical fluid is reduced in pressure (8). The pressure drop during the reduction in pressure is preferably between 2.03 and 30.38 MPa. Simultaneously, the active substance to be coated in the form of separate moving pulverulent solid particles is conveyed via a pneumatic device (7) and coinjected, preferably coaxially, with the pressure-reduced coating agent/supercritical or subcritical fluid mixture (8). In this coaxial injection, the coating agent/supercritical or subcritical fluid mixture can, for example, be outside (FIG. 3). The coated particles are recovered in a gas/solid separation filter (9), preferably at atmospheric pressure. If the CO₂ is the carrier gas (6) during the pneumatic transportation, it is possible to envisage reusing it in a closed loop (FIG. 2).

FIGS. 3 and 4 represent the principle of bringing the separate moving particles of active substance (10) and the coating agent/supercritical or subcritical fluid mixture (11) into contact by coaxial injection. The arrival of the coating agent/supercritical or subcritical fluid mixture is shown diagrammatically in (12). This mixture is then reduced in pressure (13) and thus forms an aerosol (14) which meets up with the external pipe as exemplified in (11) (FIG. 4).

The active substance to be coated is introduced in the form of separate moving particles (15) and meets up with the internal pipe (10). The contacting operation is carried out in the chamber (16), where a pressure prevails which is sufficient to allow the fluid to return to the gaseous state and to allow the coating agent to solidify around the separate particles of active substance. The coated particles are finally collected in a gas/solid separation filter (9).

This method of carrying out the contacting operation is not exclusive and alternative forms or extensions can be envisaged. The contacting time can, for example, be increased by modifying the path of the stream transporting the coating agent/supercritical or subcritical fluid mixture and the separate moving particles of active substance to be coated: instead of being linear, it can be helical. The contacting operation can also be improved by recirculation of the uncoated separate particles of active substance after separation of the latter from the coated particles (FIG. 1).

The following examples are given by way of indication and without implied limitation.

The coating of the particles is characterized by particle size determination by the dry route (equipment used: Aerosizer® PSD 3603, TSI) which makes it possible to determine the particle size distribution from the time of flight of the particles in a stream of air. The coated particles are viewed with an ESEM, Environmental Scanning Electron Microscope (Philips, XL 30 FEG) (FIGS. 7 to 10, 12, 16 and 17).

The process according to the present invention can be carried out according to the following examples:

### EXAMPLE 1

Supercritical CO₂ is dissolved at 50° C. and 11.14 MPa in 3 g of Precirol® ATO5 (supplied by Gattefossé) in the solid form (glyceryl dipalmitate/stearate, melting point=56.3° C.). The pressure in the autoclave is kept constant. After 30 minutes, the saturated Precirol® phase is reduced in pressure towards the axial coinjection system. At the same time, 3 g of glass beads with a particle size of 50-63 μm are coinjected, according to the configuration of FIG. 1, via a venturi, the compressed air pressure of which reaches 0.51 MPa. The glass beads move inside two coaxial pipes, while the sprayed fatty substance is admitted into the external pipe. This configuration makes possible favored deposition of the coating agent at the surface of the glass particles. The combined product is collected in a gas/solid separator at atmospheric pressure. The CO₂/compressed air gas mixture is conveyed to the vent. The coated particles collected are analyzed by laser particle sizing in order to monitor the increase in the mean diameter after coating. The experiment is carried out twice (Test 1 and Test 2) to confirm the reproducibility of the results. The coating is monitored by particle size determination using a PSD3603 particle size distribution analyzer (TSI) (FIGS. 5 and 6) and with an ESEM image (FIGS. 7, 8, 9 and 10) (Environmental Scanning Electron Microscope). A coating representative of Precirol® on a glass bead obtained by the process according to the present invention is shown in FIG. 8. The coating of FIG. 8 is homogeneous and is completely different from the simple physical mixing of the two components (FIG. 9) and from the surface appearance of an untreated glass bead (FIG. 7). FIG. 10 shows a group of particles obtained by coating according to the present invention. This group shows the homogeneity of the sample of the coated particles.

This shows that the coating is not only a deposition of solid Precirol® on the glass beads but involves a phenomenon of solidification/deposition on the surface of the bead. FIGS. 5 and 6 make it possible to observe a shift in the mean geometrical diameter towards greater diameters of the coated particles in comparison with the uncoated particles. This growth in the mean diameter of the particles can be attributed to the deposition of a coating layer at the surface of the particles.

In addition, the two particle size curves with regard to the coated particles of Tests 1 and 2 displayed in FIGS. 5 and 6 are similar and make it possible to deduce that the reproducibility of the process is very good.

### EXAMPLE 2

In this example, the starting weights of materials to be coated and of coating agent are changed. 4 g of Precirol® ATO5 fatty substance in the solid form are dissolved under the same conditions as in Example 1 but the particle size of the glass beads is restricted to a size of 30-40 μm. The amount of coating agent employed for the coating is reduced. In addition, gaseous CO₂ is used to convey the 6 g of glass beads towards the coinjection system (according to FIG. 2). The particles are collected in the collector at atmospheric pressure. The experiment is carried out twice (Test 1 and Test 2) to confirm the reproducibility of the results. The coating is monitored by particle size determination using a PSD3603 particle size distribution analyzer (TSI) (FIG. 11) and with an ESEM (Environmental Scanning Electron Microscope) image (FIG. 12). FIG. 11 makes it possible to observe a shift in the mean diameter of the particles towards greater diameters. The increase in the diameter of the coated particles with respect to the diameter of the uncoated particles is a consequence of the deposition of coating agent at the surface of the particles. FIG. 12 is an ESEM photograph, representative of the group obtained, of a glass bead coated with Precirol®.

### EXAMPLE 3

Supercritical CO₂ is dissolved at 50° C. and 11.14 MPa in 3 g of Precirol® ATO5 in the solid form (glyceryl dipalmitate/stearate, melting point=56.3° C.). The pressure in the autoclave is kept constant. After 30 minutes, the Precirol® ATO5, saturated with CO₂, is reduced in pressure towards the axial coinjection system. At the same time, 3 g of pseudoephedrine particles with a mean diameter by volume of 14.9±1.4 μm are coinjected according to the configuration of FIG. 2. The pressure in the autoclave via which the injection of the pseudoephedrine is carried out is set at 5.06 MPa. They move inside the central pipe of the coaxial injection, while the sprayed fatty substance is admitted into the external pipe. The combined product is collected in a gas/solid separator at atmospheric pressure. The experiment is carried out twice to confirm the reproducibility of the results (Tests 1 and 2). The coated particles collected are analyzed by laser particle sizing using a PSD3603 particle size distribution analyzer (TSI) to monitor the increase in the mean diameter after coating. The cumulative distributions by volume of the pseudoephedrine alone and of the coated pseudoephedrine are given in FIG. 13. The shift in the distribution towards greater sizes, a phenomenon due to the coating and to the agglomeration of the particles, is clearly seen.

The dissolution tests carried out in distilled water at ambient temperature show that the coating is effective since a marked delay is observed in the release of the active compound (FIG. 14). The coated particles exhibit a delay in the release in comparison with uncoated pseudoephedrine.

### EXAMPLE 4

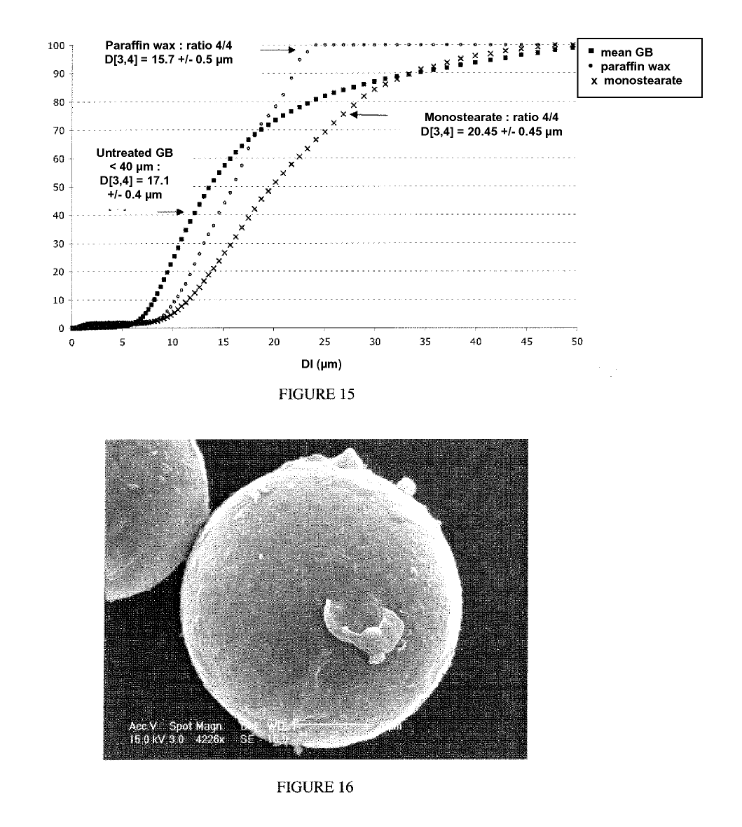

The coating of glass bead particles with a particle size of less than 20 μm is carried out this time. Supercritical CO₂ is dissolved under the conditions of a pressure of 11.14 MPa and of a temperature of 53° C. in 4 g of paraffin wax used for the coating of pharmaceutical compounds. Gaseous CO₂ is used at a pressure of 8 MPa to convey the 6 g of glass beads towards the coinjection system (according to FIG. 2). After a dissolution time of 30 min, the uncoated particles are conveyed by the gaseous CO₂ and, simultaneously, the paraffin wax is crystallized by opening the valve at the outlet of the dissolution chamber. The glass beads move inside two coaxial pipes, while the sprayed fatty substance is conveyed in the external pipe. The particles are collected in the collector at atmospheric pressure. The coating is monitored by particle size determination using a PSD3603 particle size distribution analyzer (TSI) (FIG. 15) and with an ESEM (Environmental Scanning Electron Microscope) image (FIG. 16). FIG. 15 makes it possible to observe a shift in the mean diameter of the particles towards greater diameters. The increase in the diameter of the coated particles with respect to the diameter of the uncoated particles is a consequence of the deposition of coating agent at the surface of the particles. FIG. 16 makes it possible to view the coating of a representative glass bead particle. A continuous layer of coating agent is seen at the surface of the glass bead. This example shows that the process can be applied to particles with a particle size of less than 20 μm.

### EXAMPLE5

Glass bead particles with a particle size of less than 20 μm are again coated. This time, supercritical CO₂ is dissolved under the conditions of a pressure of 11.14 MPa and of a temperature of 57.3° C. in 4 g of glyceryl monostearate used as coating agent in the pharmaceutical industry. Gaseous CO₂ is used at a pressure of 8 MPa to convey the 6 g of glass beads towards the coinjection system (according to FIG. 2). The implementation is similar to that of Example 4. The coating is monitored by particle size determination using a PSD3603 particle size distribution analyzer (TSI) (FIG. 15) and with an ESEM (Environmental Scanning Electron Microscope) image (FIG. 17). FIG. 15 makes it possible to observe a shift in the mean diameter of the particles coated with the monostearate towards greater diameters. FIG. 17 makes it possible to view the layer of coating deposit at the surface of the glass bead particles.

### EXAMPLE 6

The claimed process is carried out according to FIG. 2 in order to coat pseudoephedrine with glyceryl monostearate, the melting point of which at atmospheric pressure is 60.3° C. The supercritical CO₂ is dissolved at a temperature of 57° C. and at a pressure of 110 bar in 4 g of the fatty substance used for the coating. After an equilibrium time of 30 minutes, the heavy phase of liquefied fatty substance and of dissolved CO₂ is reduced in pressure. Simultaneously, 4 g of pseudo-ephedrine are injected via CO₂ at a pressure of 80 bar. The particles are collected in a solid/gas separator for analysis. The quantitative determination by UV spectrophotometry of the coated pseudoephedrine released into distilled water at ambient temperature is presented in FIG. 18. The crude pseudoephedrine is completely dissolved in solution in 10 minutes. The pseudoephedrine present in the coated particles takes 60 minutes to dissolve in the aqueous medium, i.e. a delay in the dissolution of close to 50 minutes.

### EXAMPLE 7

Example 7 presents the coating of 4 g of pseudoephedrine by 4 g of paraffin wax, the melting point of which at atmospheric pressure is 58.5° C. The conditions for dissolution of the supercritical CO₂ in the fatty substance are 110 bar and 55° C. The dissolution time is 30 minutes. After the dissolution stage, the two streams, material to be coated and coating agent, are coinjected according to FIG. 2 of the process according to the present invention. The coated particles are quantitatively determined by UV spectrophotometry in order to locate the delay effect in the dissolution caused by the coating of the particles (FIG. 18). It is noticed, in FIG. 18, that the dissolution in distilled water at ambient temperature of the active principle is delayed by approximately 50 minutes by the coating with respect to the crude pseudoephedrine.

### EXAMPLE 8

This time, a heat-sensitive protein, BSA (Bovine Serum Albumin), the mean diameter of which, measured by particle size determination, is 85.6 μm, is coated according to FIG. 2 of the process according to the present invention. 3 g of BSA are placed in the system for injection by CO₂ and 3 g of Precirol® are placed in the autoclave. The supercritical medium conditions are 110 bar and 50° C. for a dissolution time of 30 minutes. The pressure for injection of the BSA particles is 80 bar. Once coinjection has been carried out, the coated particles are quantitatively determined by UV spectrophotometry (FIG. 19). While the crude BSA is completely dissolved in distilled water at ambient temperature after 5 minutes, the protein coated with Precirol® (BSA 1 test) for its part takes close to 30 minutes in order to be completely dissolved in distilled water at ambient temperature.

1. Coated microparticles of active substance, each microparticle successively comprising a core comprising the active substance and a shell comprising the coating agent, wherein
1) they are coated particles in the form of fine moving separate particles which are not agglomerated;
2) the active substance is solid at ambient temperature and is sensitive to heat;
3) the mean size of the core of active substance is less than 50 μm; and
4) the coating agent is insoluble in the supercritical or subcritical fluid.

2. Coated particles of active substance, each particle successively comprising a core comprising the active substance and a shell comprising the coating agent, wherein
1) they are coated particles in the form of fine moving separate particles which are not agglomerated,
2) the active substance is solid at ambient temperature and is sensitive to heat,
3) the mean size of the core of active substance is less than 100 μm,
4) the coating agent is solid at ambient temperature and is insoluble in the supercritical or subcritical fluid, and
5) the coated particles are devoid of organic solvent.

3. Particles according to claim 1 or 2, wherein the coating agent makes it possible to mask the taste and/or color of the active substance.

4. Particles according to claim 1 or 2, wherein the coating agent makes possible controlled release of the active substance and an increase in its bioavailability.

5. Particles according to claim 1 or 2, wherein the coating agent makes it possible to protect the active substance with regard to external agents which cause damage.

6. Particles according to claim 4, wherein the active substance is sensitive to chemicals or to light.

7. Oral, topical, injectable or rectal formulation which contains the particles according to claim 1 or 2.

8. Pharmaceutical, cosmetic, nutraceutical, food, agrochemical or veterinary formulation which contains the particles according to claim 1 or 2.

9. Coated microparticles of active substance, each microparticle successively comprising a core comprising the active substance and a shell comprising the coating agent, wherein
1) they are obtainable by the process for coating pulverulent solid active substances to form coated particles in the form of fine moving separate particles which are not agglomerated, which comprises the following stages:
a) providing a mixture comprising at least one coating agent in which is dissolved, up to saturation, a supercritical or subcritical fluid under temperature and pressure conditions which make it possible to maintain the said fluid under supercritical or subcritical conditions;
b) providing separate moving particles of at least one pulverulent solid active substance;
c) bringing the mixture and the separate moving particles of active substance into contact under temperature and pressure conditions which simultaneously ensure: the reduction in pressure of the supercritical or subcritical fluid and its return to the gaseous state; the spraying of the coating agent; and its solidification around the separate moving particles of active substance; and
d) separating and recovering the coated particles,
2) the active substance is solid at ambient temperature and is sensitive to heat,
3) the mean size of the core of active substance is less than 50 μm, and
4) the coating agent is insoluble in the supercritical or subcritical fluid.

10. Coated particles of active substance, each particle successively comprising a core comprising the active substance and a shell comprising the coating agent, wherein
1) they are obtainable by the process for coating pulverulent solid active substances to form coated particles in the form of fine moving separate particles which are not agglomerated, which comprises the following stages:
a) providing a mixture comprising at least one coating agent in which is dissolved, up to saturation, a supercritical or subcritical fluid under temperature and pressure conditions which make it possible to maintain the said fluid under supercritical or subcritical conditions;
b) providing separate moving particles of at least one pulverulent solid active substance;
c) bringing the mixture and the separate moving particles of active substance into contact under temperature and pressure conditions which simultaneously ensure: the reduction in pressure of the supercritical or subcritical fluid and its return to the gaseous state; the spraying of the coating agent and its solidification around the separate moving particles of active substance, and
d) separating and recovering the coated particles,
2) the active substance is solid at ambient temperature and is sensitive to heat,
3) the mean size of the core of active substance is less than 100 μm,
4) the coating agent is solid at ambient temperature and is soluble in the supercritical or subcritical fluid, and
5) the coated particles are devoid or organic solvent.
